(19) United States
(12) Patent Application Publication
(10) Pub. No.: US 2022/0104704 A1
(43) Pub. Date: Apr. 7, 2022

(54) Sleep Monitoring Based on Wireless Signals Received by a Wireless Communication Device

(71) Applicant: Cognitive Systems Corp., Waterloo (CA)

(72) Inventors: Mikhail Alexand Zakharov, Guelph (CA); Oleksiy Kravets, Petersburg (CA)

(73) Assignee: Cognitive Systems Corp., Waterloo (CA)

(21) Appl. No.: 17/494,314

(22) Filed: Oct. 5, 2021

Related U.S. Application Data
(60) Provisional application No. 63/087,583, filed on Oct. 5, 2020.

(51) Int. Cl. A61B 5/00; A61B 5/11; A61B 5/08

(52) U.S. Cl. CPC A61B 5/0002; A61B 5/4815; A61B 5/743; A61B 5/1118; A61B 5/0816; A61B 5/4809

(57) ABSTRACT

In a general aspect, a wireless communication device operating as a client in a wireless communication network receives wireless signals transmitted from an access point of the network. The device generates channel information from the wireless signals and processes the channel information to identify a degree of motion and average breathing rate of a person. Upon determining that the degree of motion and average breathing rate are below respective thresholds, the device begins sleep monitoring. Sleep monitoring includes generating additional channel information that is processed to identify a category of sleep.

### CROSS REFERENCE TO RELATED APPLICATIONS

This application claims priority to U.S. Provisional Patent Application No. 63/087,583, filed Oct. 5, 2020, entitled “Sensing Motion Using a Client Device.” The above-referenced priority application is hereby incorporated by reference.

### BACKGROUND

The following description relates to sleep monitoring based on wireless signals received by a wireless communication device.

Motion detection systems have been used to detect movement, for example, of objects in a room or an outdoor area. In some example motion detection systems, infrared or optical sensors are used to detect movement of objects in the sensor's field of view. Motion detection systems have been used in security systems, automated control systems and other types of systems.

### DESCRIPTION OF DRAWINGS

FIG. 1 is a diagram showing an example wireless communication system.

FIGS. 2A-2B are diagrams showing example wireless signals communicated between wireless communication devices.

FIG. 2C is a diagram showing an example wireless sensing system operating to detect motion in a space.

FIG. 3 is a diagram showing an example graphical display on a user interface on a user device.

FIG. 4 is a diagram showing an example client device operating to determine the breathing rate and sleeping behavior of a person in a space.

FIG. 5 is a diagram showing example changes in channel information over time that can be used by a client device to determine the breathing rate of a person.

FIG. 6 is a diagram showing a plot of a degree of motion as a function of time and a plot showing corresponding periods of disrupted, light, and restful sleep.

FIGS. 7A and 7B are diagrams showing example implementations of client devices having a motion detection system.

FIG. 8 is a block diagram showing an example wireless communication device.

### DETAILED DESCRIPTION

In some aspects of what is described here, a wireless sensing system can process wireless signals (e.g., radio frequency signals) transmitted through a space between wireless communication devices for wireless sensing applications. Example wireless sensing applications include detecting motion, which can include one or more of the following: detecting motion of objects in the space, motion tracking, localization of motion in a space, breathing detection, breathing monitoring, presence detection, gesture detection, gesture recognition, human detection (e.g., moving and stationary human detection), human tracking, fall detection, speed estimation, intrusion detection, walking detection, step counting, respiration rate detection, sleep pattern detection, sleep quality monitoring, apnea estimation, posture change detection, activity recognition, gait rate classification, gesture decoding, sign language recognition, hand tracking, heart rate estimation, breathing rate estimation, room occupancy detection, human dynamics monitoring, and other types of motion detection applications. Other examples of wireless sensing applications include object recognition, speaking recognition, keystroke detection and recognition, tamper detection, touch detection, attack detection, user authentication, driver fatigue detection, traffic monitoring, smoking detection, school violence detection, human counting, metal detection, human recognition, bike localization, human queue estimation, Wi-Fi imaging, and other types of wireless sensing applications. For instance, the wireless sensing system may operate as a motion detection system to detect the existence and location of motion based on Wi-Fi signals or other types of wireless signals.

The examples described herein may be useful for home monitoring. Home monitoring using the wireless sensing systems described herein provide several advantages, including full home coverage through walls and in darkness, discreet detection without cameras, higher accuracy and reduced false alerts (e.g., in comparison with sensors that do not use Wi-Fi signals to sense their environments), and adjustable sensitivity. By layering Wi-Fi motion detection capabilities into routers and gateways, a robust motion detection system may be provided.

The examples described herein may also be useful for wellness monitoring. Caregivers want to know their loved ones are safe, while seniors and people with special needs want to maintain their independence at home with dignity. Wellness monitoring using the wireless sensing systems described herein provide a solution that uses wireless signals to detect motion without using cameras or infringing on privacy, generates alerts when unusual activity is detected, tracks sleep patterns, and generates preventative health data. For example, caregivers can monitor motion, visits from health care professionals, and unusual behavior such as staying in bed longer than normal. Furthermore, motion is monitored unobtrusively without the need for wearable devices, and the wireless sensing systems described herein offer a more affordable and convenient alternative to assisted living facilities and other security and health monitoring tools.

The examples described herein may also be useful for setting up a smart home. In some examples, the wireless sensing systems described herein use predictive analytics and artificial intelligence (AI), to learn motion patterns and trigger smart home functions accordingly. Examples of smart home functions that may be triggered included adjusting the thermostat when a person walk through the front door, turning other smart devices on or off based on preferences, automatically adjusting lighting, adjusting HVAC systems based on present occupants, etc.

In some aspects of what is described here, a client device is used to identify a category of sleep of a person by monitoring sleep motion in a space using a motion detection system installed on the client device. The client device may be a Wi-Fi client device (e.g., a smartphone or wearable device like a smartwatch). In some implementations, an access point device in the space is alleviated from the need of having a motion detection system installed thereon, therefore allowing the access point device to be dedicated for providing wireless access to the client device and to other wireless-enabled devices in the space. In some implementations, the access point device provides Wi-Fi access point capabilities through SSID broadcasts that can be used by the client device as a source for channel information. In some instances, the motion detection system can be installed as a user application on the client device, as part of the client device's operating system, or otherwise. The motion detection system may have access to channel information (e.g., Wi-Fi channel state information data) provided by radio firmware of the client device, for example, to sense motion in the space.

In some implementations, the channel information can be obtained by the client device through passive sensing by capturing periodic broadcast information from one or more access point devices (e.g., SSID broadcasts). In some implementations, the channel information can be obtained by the client device through active sounding from the client device to its associated access point device. In such implementations, the access point device responds to request frames from the client device and the corresponding response is received and processed by the client device. In some implementations, the channel information can be obtained through pre-existing data traffic between the client device and the access point device. In some implementations, the channel information can be obtained through the client device being in a “promiscuous mode” when the client device eavesdrops on wireless traffic between other devices in the wireless network. The channel information can be obtained by the client device on a regular basis (e.g., multiple times per second) and corresponding motion detection or localization algorithms can process the channel information over time to extract information about characteristics of the changing physical environment in proximity to the client device. The output of the motion detection system may include data that is indicative of motion.

In some implementations, information about detected motion, respiratory activity, sleep monitoring can be provided to a user in the form of a mobile application user interface, notifications, and audio or video alerts provided by the client device itself or other devices with user interface capabilities. Notification to a designated emergency contact or caregiver can be provided as well. In some implementations, the channel information may be processed on the client device itself, or the channel information can be sent to the cloud server to be processed remotely. Information to the end-user may be provided in real-time form (e.g., active monitoring results calculated with minimal possible latency—typically in the order of few seconds), as statistical information calculated over a longer time (e.g., hours or days), or both.

In some instances, aspects of the systems and techniques described here provide technical improvements and advantages over existing approaches. For example, use of a client device (e.g., instead of an access point device) to detect motion may allow a wireless sensing system to utilize a broad range of wireless communication devices for wireless sensing, to operate in more diverse environments, to cover greater spatial areas, to leverage existing hardware (e.g., which may reduce or eliminate a requirement for specialized motion detection hardware in some cases), or to provide a combination of these and other advantages. The technical improvements and advantages achieved in examples where the wireless sensing system is used for motion detection may also be achieved in other examples where the wireless sensing system is used for other wireless sensing applications.

In some instances, a wireless sensing system can be implemented using a wireless communication network. Wireless signals received at one or more wireless communication devices in the wireless communication network may be analyzed to determine channel information for the different communication links (between respective pairs of wireless communication devices) in the network. The channel information may be representative of a physical medium that applies a transfer function to wireless signals that traverse a space. In some instances, the channel information includes a channel response. Channel responses can characterize a physical communication path, representing the combined effect of, for example, scattering, fading, and power decay within the space between the transmitter and receiver. In some instances, the channel information includes beamforming state information (e.g., a feedback matrix, a steering matrix, channel state information (CSI), etc.) provided by a beamforming system. Beamforming is a signal processing technique often used in multi antenna (multiple-input/multiple-output (MIMO)) radio systems for directional signal transmission or reception. Beamforming can be achieved by operating elements in an antenna array in such a way that signals at particular angles experience constructive interference while others experience destructive interference.

The channel information for each of the communication links may be analyzed by one or more motion detection or localization algorithms (e.g., running on a hub device, a client device, or other device in the wireless communication network, or on a remote device communicably coupled to the network) to detect, for example, whether motion has occurred in the space, to determine a relative location of the detected motion, or both. In some aspects, the channel information for each of the communication links may be analyzed to detect whether an object is present or absent, e.g., when no motion is detected in the space.

In some instances, a motion detection system returns motion data. In some implementations, motion data is a result that is indicative of a degree of motion in the space, the location of motion in the space, a time at which the motion occurred, or a combination thereof. In some instances, motion data may include an indication of a person's breathing rate, an indication or classification of a person's sleeping behavior, or both. In some instances, the motion data can include a motion score, which may include, or may be, one or more of the following: a scalar quantity indicative of a level of signal perturbation in the environment accessed by the wireless signals; an indication of whether there is motion; an indication of whether there is an object present; or an indication or classification of a gesture performed in the environment accessed by the wireless signals.

In some implementations, the motion detection system can be implemented using motion detection or localization algorithms. Example motion detection or localization algorithms that can be used to detect motion based on wireless signals include the techniques described in U.S. Pat. No. 9,523,760 entitled “Detecting Motion Based on Repeated Wireless Transmissions,” U.S. Pat. No. 9,584,974 entitled “Detecting Motion Based on Reference Signal Transmissions,” U.S. Pat. No. 10,051,414 entitled “Detecting Motion Based On Decompositions Of Channel Response Variations,” U.S. Pat. No. 10,048,350 entitled “Motion Detection Based on Groupings of Statistical Parameters of Wireless Signals,” U.S. Pat. No. 10,108,903 entitled “Motion Detection Based on Machine Learning of Wireless Signal Properties,” U.S. Pat. No. 10,109,167 entitled “Motion Localization in a Wireless Mesh Network Based on Motion Indicator Values,” U.S. Pat. No. 10,109,168 entitled “Motion Localization Based on Channel Response Characteristics,” U.S. Pat. No. 10,743,143 entitled “Determining a Motion Zone for a Location of Motion Detected by Wireless Signals,” U.S. Pat. No. 10,605,908 entitled “Motion Detection Based on Beamforming Dynamic Information from Wireless Standard Client Devices,” U.S. Pat. No. 10,605,907 entitled “Motion Detection by a Central Controller Using Beamforming Dynamic Information,” U.S. Pat. No. 10,600,314 entitled “Modifying Sensitivity Settings in a Motion Detection System,” U.S. Pat. No. 10,567,914 entitled “Initializing Probability Vectors for Determining a Location of Motion Detected from Wireless Signals,” U.S. Pat. No. 10,565,860 entitled “Offline Tuning System for Detecting New Motion Zones in a Motion Detection System,” U.S. Pat. No. 10,506,384 entitled “Determining a Location of Motion Detected from Wireless Signals Based on Prior Probability, ” U.S. Pat. No. 10,499,364 entitled “Identifying Static Leaf Nodes in a Motion Detection System,” U.S. Pat. No. 10,498,467 entitled “Classifying Static Leaf Nodes in a Motion Detection System,” U.S. Pat. No. 10,460,581 entitled “Determining a Confidence for a Motion Zone Identified as a Location of Motion for Motion Detected by Wireless Signals,” U.S. Pat. No. 10,459,076 entitled “Motion Detection based on Beamforming Dynamic Information,” U.S. Pat. No. 10,459,074 entitled “Determining a Location of Motion Detected from Wireless Signals Based on Wireless Link Counting,” U.S. Pat. No. 10,438,468 entitled “Motion Localization in a Wireless Mesh Network Based on Motion Indicator Values,” U.S. Pat. No. 10,404,387 entitled “Determining Motion Zones in a Space Traversed by Wireless Signals,” U.S. Pat. No. 10,393,866 entitled “Detecting Presence Based on Wireless Signal Analysis,” U.S. Pat. No. 10,380,856 entitled “Motion Localization Based on Channel Response Characteristics,” U.S. Pat. No. 10,318,890 entitled “Training Data for a Motion Detection System using Data from a Sensor Device,” U.S. Pat. No. 10,264,405 entitled “Motion Detection in Mesh Networks,” U.S. Pat. No. 10,228,439 entitled “Motion Detection Based on Filtered Statistical Parameters of Wireless Signals,” U.S. Pat. No. 10,129,853 entitled “Operating a Motion Detection Channel in a Wireless Communication Network,” U.S. Pat. No. 10,111,228 entitled “Selecting Wireless Communication Channels Based on Signal Quality Metrics,” and other techniques.

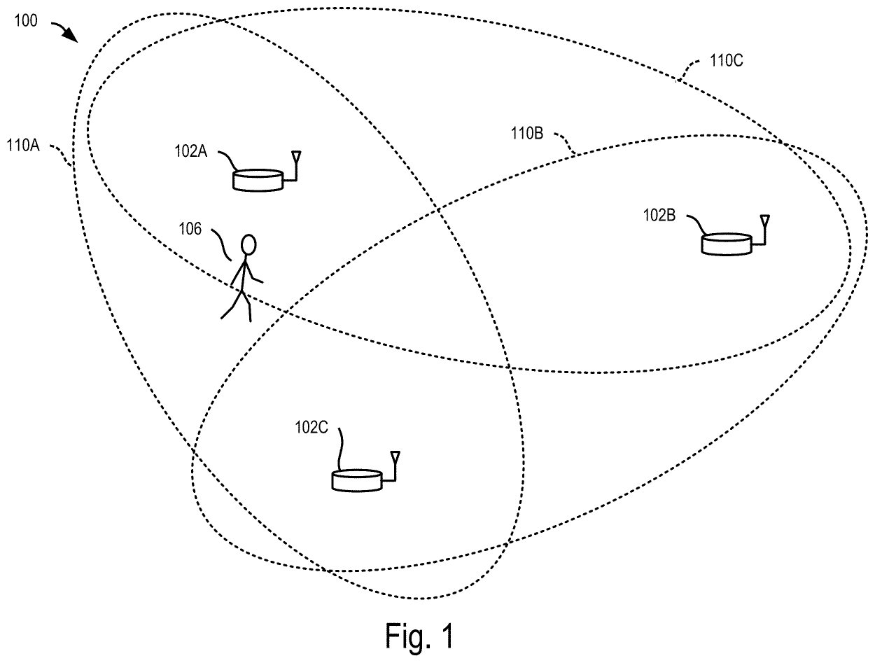

FIG. 1 illustrates an example wireless communication system 100. The wireless communication system 100 may perform one or more operations of a motion detection system. The technical improvements and advantages achieved from using the wireless communication system 100 to detect motion are also applicable in examples where the wireless communication system 100 is used for another wireless sensing application.

The example wireless communication system 100 includes three wireless communication devices 102A, 102B, 102C. The example wireless communication system 100 may include additional wireless communication devices 102 and/or other components (e.g., one or more network servers, network routers, network switches, cables, or other communication links, etc.).

The example wireless communication devices 102A, 102B, 102C can operate in a wireless network, for example, according to a wireless network standard or another type of wireless communication protocol. For example, the wireless network may be configured to operate as a Wireless Local Area Network (WLAN), a Personal Area Network (PAN), a metropolitan area network (MAN), or another type of wireless network. Examples of WLANs include networks configured to operate according to one or more of the 802.11 family of standards developed by IEEE (e.g., Wi-Fi networks), and others. Examples of PANs include networks that operate according to short-range communication standards (e.g., BLUETOOTH®, Near Field Communication (NFC), ZigBee), millimeter wave communications, and others.

In some implementations, the wireless communication devices 102A, 102B, 102C may be configured to communicate in a cellular network, for example, according to a cellular network standard. Examples of cellular networks include networks configured according to 2G standards such as Global System for Mobile (GSM) and Enhanced Data rates for GSM Evolution (EDGE) or EGPRS; 3G standards such as Code Division Multiple Access (CDMA), Wideband Code Division Multiple Access (WCDMA), Universal Mobile Telecommunications System (UMTS), and Time Division Synchronous Code Division Multiple Access (TD-SCDMA); 4G standards such as Long-Term Evolution (LTE) and LTE-Advanced (LTE-A); 5G standards, and others.

In some cases, one or more of the wireless communication devices 102 is a Wi-Fi access point or another type of wireless access point (WAP). In some cases, one or more of the wireless communication devices 102 is an access point of a wireless mesh network, such as, for example, a commercially-available mesh network system (e.g., GOOGLE Wi-Fi, EERO mesh, etc.). In some instances, one or more of the wireless communication devices 102 can be implemented as wireless access points (APs) in a mesh network, while the other wireless communication device(s) 102 are implemented as leaf devices (e.g., mobile devices, smart devices, etc.) that access the mesh network through one of the APs. In some cases, one or more of the wireless communication devices 102 is a mobile device (e.g., a smartphone, a smart watch, a tablet, a laptop computer, etc.), a wireless-enabled device (e.g., a smart thermostat, a Wi-Fi enabled camera, a smart TV), or another type of device that communicates in a wireless network.

In the example shown in FIG. 1, the wireless communication devices transmit wireless signals to each other over wireless communication links (e.g., according to a wireless network standard or a non-standard wireless communication protocol), and the wireless signals communicated between the devices can be used as motion probes to detect motion of objects in the signal paths between the devices. In some implementations, standard signals (e.g., channel sounding signals, beacon signals), non-standard reference signals, or other types of wireless signals can be used as motion probes.

In the example shown in FIG. 1, the wireless communication link between the wireless communication devices 102A, 102C can be used to probe a first motion detection zone 110A, the wireless communication link between the wireless communication devices 102B, 102C can be used to probe a second motion detection zone 110B, and the wireless communication link between the wireless communication device 102A, 102B can be used to probe a third motion detection zone 110C. In some instances, the motion detection zones 110 can include, for example, air, solid materials, liquids, or another medium through which wireless electromagnetic signals may propagate.

In the example shown in FIG. 1, when an object moves in any of the motion detection zones 110, the motion detection system may detect the motion based on signals transmitted through the relevant motion detection zone 110. Generally, the object can be any type of static or moveable object, and can be living or inanimate. For example, the object can be a human (e.g., the person 106 shown in FIG. 1), an animal, an inorganic object, or another device, apparatus, or assembly, an object that defines all or part of the boundary of a space (e.g., a wall, door, window, etc.), or another type of object.

In some examples, the wireless signals may propagate through a structure (e.g., a wall) before or after interacting with a moving object, which may allow the object's motion to be detected without an optical line-of-sight between the moving object and the transmission or receiving hardware. In some instances, the motion detection system may communicate the motion detection event to another device or system, such as a security system or a control center.

In some cases, the wireless communication devices 102 themselves are configured to perform one or more operations of the motion detection system, for example, by executing computer-readable instructions (e.g., software or firmware) on the wireless communication devices. For example, each device may process received wireless signals to detect motion based on changes in the communication channel. In some cases, another device (e.g., a remote server, a cloud-based computer system, a network-attached device, etc.) is configured to perform one or more operations of the motion detection system. For example, each wireless communication device 102 may send channel information to a specified device, system or service that performs operations of the motion detection system.

In an example aspect of operation, wireless communication devices 102A, 102B may broadcast wireless signals or address wireless signals to the other wireless communication device 102C, and the wireless communication device 102C (and potentially other devices) receives the wireless signals transmitted by the wireless communication devices 102A, 102B. The wireless communication device 102C (or another system or device) then processes the received wireless signals to detect motion of an object in a space accessed by the wireless signals (e.g., in the zones 110A, 11B). In some instances, the wireless communication device 102C (or another system or device) may perform one or more operations of a motion detection system.

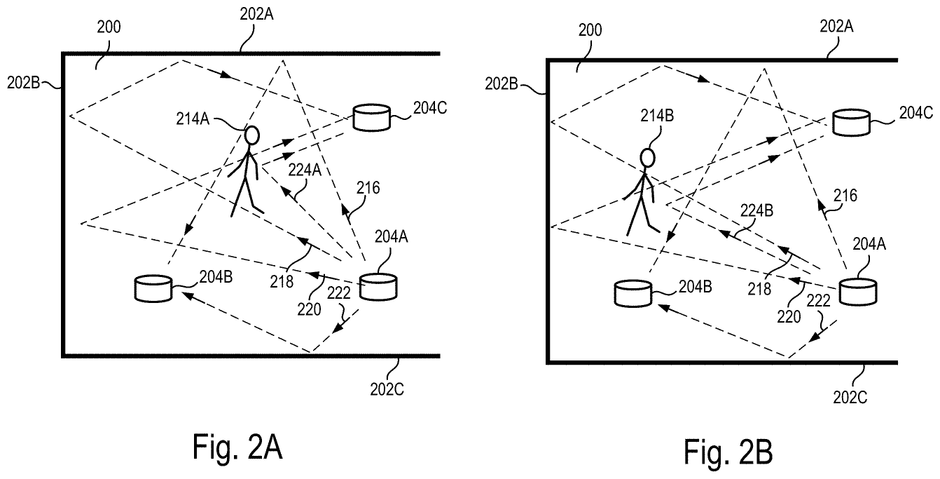

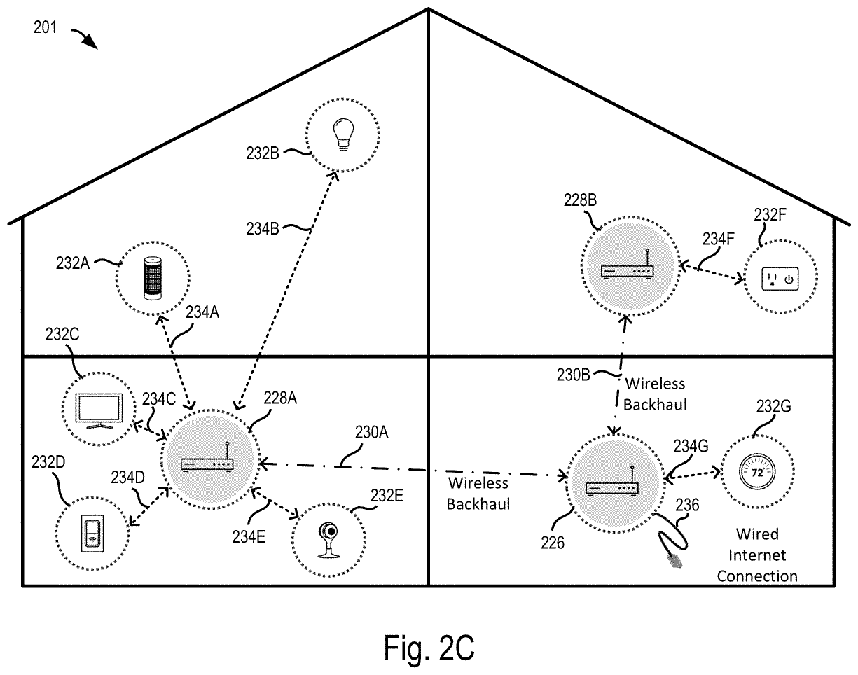

FIGS. 2A and 2B are diagrams showing example wireless signals communicated between wireless communication devices 204A, 204B, 204C. The wireless communication devices 204A, 204B, 204C may be, for example, the wireless communication devices 102A, 102B, 102C shown in FIG. 1, or may be other types of wireless communication devices.

In some cases, a combination of one or more of the wireless communication devices 204A, 204B, 204C can be part of, or may be used by, a motion detection system. The example wireless communication devices 204A, 204B, 204C can transmit wireless signals through a space 200. The example space 200 may be completely or partially enclosed or open at one or more boundaries of the space 200. The space 200 may be or may include an interior of a room, multiple rooms, a building, an indoor area, outdoor area, or the like. A first wall 202A, a second wall 202B, and a third wall 202C at least partially enclose the space 200 in the example shown.

In the example shown in FIGS. 2A and 2B, the first wireless communication device 204A transmits wireless motion probe signals repeatedly (e.g., periodically, intermittently, at scheduled, unscheduled or random intervals, etc.). The second and third wireless communication devices 204B, 204C receive signals based on the motion probe signals transmitted by the wireless communication device 204A.

As shown, an object is in a first position 214A at an initial time (t0) in FIG. 2A, and the object has moved to a second position 214B at subsequent time (t1) in FIG. 2B. In FIGS. 2A and 2B, the moving object in the space 200 is represented as a human, but the moving object can be another type of object. For example, the moving object can be an animal, an inorganic object (e.g., a system, device, apparatus, or assembly), an object that defines all or part of the boundary of the space 200 (e.g., a wall, door, window, etc.), or another type of object. In the example shown in FIGS. 2A and 2B, the wireless communication devices 204A, 204B, 204C are stationary and are, consequently, at the same position at the initial time t0 and at the subsequent time t1. However, in other examples, one or more of the wireless communication devices 204A, 204B, 204C may be mobile and may move between initial time t0 and subsequent time t1.

As shown in FIGS. 2A and 2B, multiple example paths of the wireless signals transmitted from the first wireless communication device 204A are illustrated by dashed lines. Along a first signal path 216, the wireless signal is transmitted from the first wireless communication device 204A and reflected off the first wall 202A toward the second wireless communication device 204B. Along a second signal path 218, the wireless signal is transmitted from the first wireless communication device 204A and reflected off the second wall 202B and the first wall 202A toward the third wireless communication device 204C. Along a third signal path 220, the wireless signal is transmitted from the first wireless communication device 204A and reflected off the second wall 202B toward the third wireless communication device 204C. Along a fourth signal path 222, the wireless signal is transmitted from the first wireless communication device 204A and reflected off the third wall 202C toward the second wireless communication device 204B.

In FIG. 2A, along a fifth signal path 224A, the wireless signal is transmitted from the first wireless communication device 204A and reflected off the object at the first position 214A toward the third wireless communication device 204C. Between time t0 in FIG. 2A and time t1 in FIG. 2B, the object moves from the first position 214A to a second position 214B in the space 200 (e.g., some distance away from the first position 214A). In FIG. 2B, along a sixth signal path 224B, the wireless signal is transmitted from the first wireless communication device 204A and reflected off the object at the second position 214B toward the third wireless communication device 204C. The sixth signal path 224B depicted in FIG. 2B is longer than the fifth signal path 224A depicted in FIG. 2A due to the movement of the object from the first position 214A to the second position 214B. In some examples, a signal path can be added, removed, or otherwise modified due to movement of an object in a space.

The example wireless signals shown in FIGS. 2A and 2B may experience attenuation, frequency shifts, phase shifts, or other effects through their respective paths and may have portions that propagate in another direction, for example, through the walls 202A, 202B, and 202C. In some examples, the wireless signals are radio frequency (RF) signals. The wireless signals may include other types of signals.

The transmitted signal may have a number of frequency components in a frequency bandwidth, and the transmitted signal may include one or more bands within the frequency bandwidth. The transmitted signal may be transmitted from the first wireless communication device 204A in an omnidirectional manner, in a directional manner or otherwise. In the example shown, the wireless signals traverse multiple respective paths in the space 200, and the signal along each path may become attenuated due to path losses, scattering, reflection, or the like and may have a phase or frequency offset.

As shown in FIGS. 2A and 2B, the signals from various paths 216, 218, 220, 222, 224A, and 224B combine at the third wireless communication device 204C and the second wireless communication device 204B to form received signals. Because of the effects of the multiple paths in the space 200 on the transmitted signal, the space 200 may be represented as a transfer function (e.g., a filter) in which the transmitted signal is input and the received signal is output. When an object moves in the space 200, the attenuation or phase offset applied to a wireless signal along a signal path can change, and hence, the transfer function of the space 200 can change. When the same wireless signal is transmitted from the first wireless communication device 204A, if the transfer function of the space 200 changes, the output of that transfer function, e.g. the received signal, can also change. A change in the received signal can be used to detect motion of an object. Conversely, in some cases, if the transfer function of the space does not change, the output of the transfer function—the received signal—may not change.

FIG. 2C is a diagram showing an example wireless sensing system operating to detect motion in a space 201. The example space 201 shown in FIG. 2C is a home that includes multiple distinct spatial regions or zones. In the example shown, the wireless motion detection system uses a multi-AP home network topology (e.g., mesh network or a Self-Organizing-Network (SON)), which includes three access points (APs): a central access point 226 and two extension access points 228A, 228B. In a typical multi-AP home network, each AP typically supports multiple bands (2.4G, 5G, 6G), and multiple bands may be enabled at the same time. Each AP may use a different Wi-Fi channel to serve its clients, as this may allow for better spectrum efficiency.

In the example shown in FIG. 2C, the wireless communication network includes a central access point 226. In a multi-AP home Wi-Fi network, one AP may be denoted as the central AP. This selection, which is often managed by manufacturer software running on each AP, is typically the AP that has a wired Internet connection 236. The other APs 228A, 228B connect to the central AP 226 wirelessly, through respective wireless backhaul connections 230A, 230B. The central AP 226 may select a wireless channel different from the extension APs to serve its connected clients.

In the example shown in FIG. 2C, the extension APs 228A, 228B extend the range of the central AP 226, by allowing devices to connect to a potentially closer AP or different channel. The end user need not be aware of which AP the device has connected to, as all services and connectivity would generally be identical. In addition to serving all connected clients, the extension APs 228A, 228B connect to the central AP 226 using the wireless backhaul connections 230A, 230B to move network traffic between other APs and provide a gateway to the Internet. Each extension AP 228A, 228B may select a different channel to serve its connected clients.

In the example shown in FIG. 2C, client devices (e.g., Wi-Fi client devices) 232A, 232B, 232C, 232D, 232E, 232F, 232G are associated with either the central AP 226 or one of the extension APs 228 using a respective wireless link 234A, 234B, 234C, 234D, 234E, 234F, 234G. The client devices 232 that connect to the multi-AP network may operate as leaf nodes in the multi-AP network. In some implementations, the client devices 232 may include wireless-enabled devices (e.g., mobile devices, a smartphone, a smart watch, a tablet, a laptop computer, a smart thermostat, a wireless-enabled camera, a smart TV, a wireless-enabled speaker, a wireless-enabled power socket, etc.).

When the client devices 232 seek to connect to and associate with their respective APs 226, 228, the client devices 232 may go through an authentication and association phase with their respective APs 226, 228. Among other things, the association phase assigns address information (e.g., an association ID or another type of unique identifier) to each of the client devices 232. For example, within the IEEE 802.11 family of standards for Wi-Fi, each of the client devices 232 may identify itself using a unique address (e.g., a 48-bit address, an example being the MAC address), although the client devices 232 may be identified using other types of identifiers embedded within one or more fields of a message. The address information (e.g., MAC address or another type of unique identifier) can be either hardcoded and fixed, or randomly generated according to the network address rules at the start of the association process. Once the client devices 232 have associated to their respective APs 226, 228, their respective address information may remain fixed. Subsequently, a transmission by the APs 226, 228 or the client devices 232 typically includes the address information (e.g., MAC address) of the transmitting wireless device and the address information (e.g., MAC address) of the receiving device.

In the example shown in FIG. 2C, the wireless backhaul connections 230A, 230B carry data between the APs and may also be used for motion detection. Each of the wireless backhaul channels (or frequency bands) may be different than the channels (or frequency bands) used for serving the connected Wi-Fi devices.

In the example shown in FIG. 2C, wireless links 234A, 234B, 234C, 234D, 234E, 234F, 234G may include a frequency channel used by the client devices 232A, 232B, 232C, 232D, 232E, 232F, 232G to communicate with their respective APs 226, 228. Each AP may select its own channel independently to serve their respective client devices, and the wireless links 234 may be used for data communications as well as motion detection.

The motion detection system, which may include one or more motion detection or localization processes running on the one or more of the client devices 232 or on one or more of the APs 226, 228, may collect and process data (e.g., channel information) corresponding to local links that are participating in the operation of the wireless sensing system. The motion detection system may be installed as a software or firmware application on the client devices 232 or on the APs 226, 228, or may be part of the operating systems of the client devices 232 or the APs 226, 228.

In some implementations, the APs 226, 228 do not contain motion detection software and are not otherwise configured to perform motion detection in the space 201. Instead, in such implementations, the operations of the motion detection system are executed on one or more of the client devices 232. In some implementations, the channel information may be obtained by the client devices 232 by receiving wireless signals from the APs 226, 228 (or possibly from other client devices 232) and processing the wireless signal to obtain the channel information. For example, the motion detection system running on the client devices 232 may have access to channel information provided by the client device's radio firmware (e.g., Wi-Fi radio firmware) so that channel information may be collected and processed.

In some implementations, the client devices 232 send a request to their corresponding AP 226, 228 to transmit wireless signals that can be used by the client device as motion probes to detect motion of objects in the space 201. The request sent to the corresponding AP 226, 228 may be a null data packet frame, a beamforming request, a ping, standard data traffic, or a combination thereof. In some implementations, the client devices 232 are stationary while performing motion detection in the space 201. In other examples, one or more of the client devices 232 may be mobile and may move within the space 201 while performing motion detection.

Mathematically, a signal f (t) transmitted from a wireless communication device (e.g., the wireless communication device 204A in FIGS. 2A and 2B or the APs 226, 228 in FIG. 2C) may be described according to Equation (1):

$$f ( t ) = {\sum }_{n = - ∞}^{∞} {c}_{n} {e}^{{j ω}_{n} t} \qquad ( 1 )$$

where ωₙ represents the frequency of n^{th} frequency component of the transmitted signal, cₙ represents the complex coefficient of the n^{th} frequency component, and t represents time. With the transmitted signal f (t) being transmitted, an output signal rₖ (t) from a path k may be described according to Equation (2):

$${r}_{k} ( t ) = {\sum }_{n = - ∞}^{∞} {α}_{n , k} {c}_{n} {e}^{j ( {ω}_{n} t + {ϕ}_{n , k} )} \qquad ( 2 )$$

where α_{n,k} represents an attenuation factor (or channel response; e.g., due to scattering, reflection, and path losses) for the n^{th} frequency component along path k, and ϕ_{n,k} represents the phase of the signal for n^{th} frequency component along path k. Then, the received signal R at a wireless communication device can be described as the summation of all output signals rₖ (t) from all paths to the wireless communication device, which is shown in Equation (3):

$$R = {\sum }_{k} {r}_{k} ( t ) \qquad ( 3 )$$

Substituting Equation (2) into Equation (3) renders the following Equation (4):

$$R = {\sum }_{k} {\sum }_{n = - ∞}^{∞} ( {α}_{n , k} {e}^{{j ϕ}_{n , k}} ) {c}_{n} {e}^{{j ω}_{n} t} \qquad ( 4 )$$

The received signal R at a wireless communication device (e.g., the wireless communication devices 204B, 204C in FIGS. 2A and 2B or the client devices 232 in FIG. 2C) can then be analyzed (e.g., using motion detection or localization algorithms) to detect motion. The received signal R at a wireless communication device can be transformed to the frequency domain, for example, using a Fast Fourier Transform (FFT) or another type of algorithm. The transformed signal can represent the received signal R as a series of n complex values, one for each of the respective frequency components (at the n frequencies ωₙ). For a frequency component at frequency ωₙ, a complex value Yₙ may be represented as follows in Equation (5):

$${Y}_{n} = {\sum }_{k} {c}_{n} {α}_{n , k} {e}^{{j ϕ}_{n , k}} . \qquad ( 5 )$$

The complex value Yₙ for a given frequency component ωₙ indicates a relative magnitude and phase offset of the received signal at that frequency component ωₙ. The signal f (t) may be repeatedly transmitted within a time period, and the complex value Yₙ can be obtained for each transmitted signal f (t). When an object moves in the space, the complex value Yₙ changes over the time period due to the channel response α_{n,k} of the space changing. Accordingly, a change detected in the channel response (and thus, the complex value Yₙ) can be indicative of motion of an object within the communication channel. Conversely, a stable channel response may indicate lack of motion. Thus, in some implementations, the complex values Yₙ for each of multiple devices in a wireless network can be processed to detect whether motion has occurred in a space traversed by the transmitted signals f (t).

In another aspect of FIGS. 2A, 2B, 2C, beamforming state information may be used to detect whether motion has occurred in a space traversed by the transmitted signals f (t). For example, beamforming may be performed between devices based on some knowledge of the communication channel (e.g., through feedback properties generated by a receiver), which can be used to generate one or more steering properties (e.g., a steering matrix) that are applied by a transmitter device to shape the transmitted beam/signal in a particular direction or directions. In some instances, changes to the steering or feedback properties used in the beamforming process indicate changes, which may be caused by moving objects in the space accessed by the wireless signals. For example, motion may be detected by identifying substantial changes in the communication channel, e.g. as indicated by a channel response, or steering or feedback properties, or any combination thereof, over a period of time.

In some implementations, for example, a steering matrix may be generated at a transmitter device (beamformer) based on a feedback matrix provided by a receiver device (beamformee) based on channel sounding. Because the steering and feedback matrices are related to propagation characteristics of the channel, these beamforming matrices change as objects move within the channel. Changes in the channel characteristics are accordingly reflected in these matrices, and by analyzing the matrices, motion can be detected, and different characteristics of the detected motion can be determined. In some implementations, a spatial map may be generated based on one or more beamforming matrices. The spatial map may indicate a general direction of an object in a space relative to a wireless communication device. In some cases, “modes” of a beamforming matrix (e.g., a feedback matrix or steering matrix) can be used to generate the spatial map. The spatial map may be used to detect the presence of motion in the space or to detect a location of the detected motion.

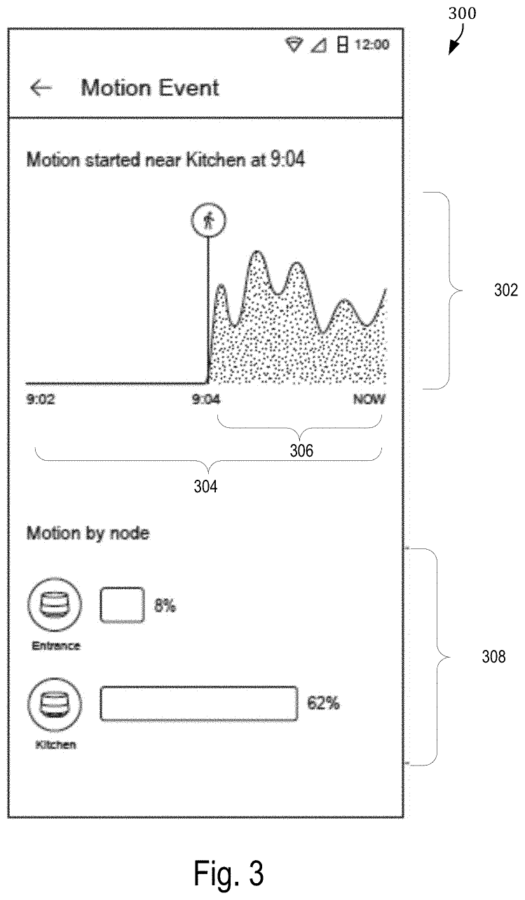

In some implementations, the output of the motion detection system may be provided as a notification for graphical display on a user interface on a user device. FIG. 3 is a diagram showing an example graphical display on a user interface 300 on a user device. In some implementations, the user device is the client device 232 used to detect motion, a user device of a caregiver or emergency contact designated to an individual in the space 200, 201, or any other user device that is communicatively coupled to the motion detection system to receive notifications from the motion detection system.

The example user interface 300 shown in FIG. 3 includes an element 302 that displays motion data generated by the motion detection system. As shown in FIG. 3, the element 302 includes a horizontal timeline that includes a time period 304 (including a series of time points 306) and a plot of motion data indicating a degree of motion detected by the motion detection system for each time point in the series of time points 306. In the example shown, the user is notified that the detected motion started near a particular location (e.g., the kitchen) at a particular time (e.g., 9:04), and the relative degree of motion detected is indicated by the height of the curve at each time point.

The example user interface 300 shown in FIG. 3 also includes an element 308 that displays the relative degree of motion detected by each node of the motion detection system. In particular, the element 308 indicates that 8% of the motion was detected by the “Entrance” node (e.g., an AP installed at the home entry) while 62% of the motion was detected by the “Kitchen” node (e.g., an AP installed in the kitchen). The data provided in the elements 302, 308 can help the user determine an appropriate action to take in response to the motion detection event, correlate the motion detection event with the user's observation or knowledge, determine whether the motion detection event was true or false, etc.

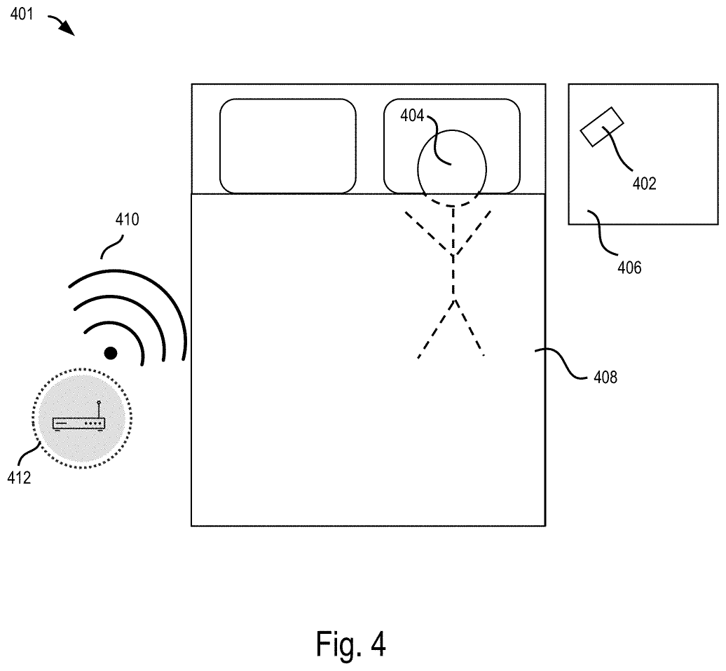

In some implementations, the output of the motion detection system may be provided in real-time (e.g., to an end user). Additionally or alternatively, the output of the motion detection system may be stored (e.g., locally on the wireless communication devices 204, client devices 232, the APs 226, 228, or on a cloud-based storage service) and analyzed to reveal statistical information over a time frame (e.g., hours, days, or months). An example where the output of the motion detection system may be stored and analyzed to reveal statistical information over a time frame is in sleep monitoring, as described with respect to FIG. 4 or otherwise. In some implementations, an alert (e.g., a notification, an audio alert, or a video alert) may be provided based on the output of the motion detection system. For example, a motion detection event may be communicated to another device or system (e.g., a security system or a control center), a designated caregiver, or a designated emergency contact based on the output of the motion detection system.

FIG. 4 is a diagram showing an example client device 402 operating to monitor motion (e.g., breathing and sleeping behavior) of a person 404 in a space 401. In the example shown in FIG. 4, the person 404 is a human being; in some cases, the client device 402 can monitor activity of multiple humans, pets, animals, etc. The client device 402 may be, for example, one or more of the client devices 232 shown in FIG. 2C, or may be other types of client devices. In the example of FIG. 4, the client device 402 is a smartphone placed on a nightstand 406 that is adjacent to a bed 408 in which the person 404 lies. In other examples, the client device 402 can be a fitness device, a smart watch, a tablet, a laptop computer, a wearable device, or any other client device located in the space 401. In some implementations, the client device 402 performs one or more operations of a motion detection system by obtaining channel information based on wireless signals 410 transmitted through the space 401 from an access point (AP) device 412 over a period of time, and detecting motion of the person 404 based on the channel information.

In some implementations, the client device 402 may be connected to (e.g., associated with) the AP device 412 via a wireless link. The client device 402 may also operate as a leaf node in a multi-AP network. In some implementations, the wireless signals 410 may be transmitted because of active sounding by the client device 402. As an example, the client device 402 may transmit, to the AP device 412, requests for the AP device 412 to transmit the wireless signals 410. The requests may include a null data packet frame, a beamforming request, a ping, or a combination thereof. In some implementations, the requests may be sent at a rate in a range from about 5 requests per second to about 15 requests per second (e.g., about 10 requests per second). The AP device 412 responds to the requests made by the client device 402 by transmitting the wireless signals 410 over a time period. The client device 402 obtains channel information based on the wireless signals 410 and detects motion of the person 404 based on the channel information.

In some implementations, the wireless signals 410 may include, or may be, pre-existing data traffic between the AP device 412 and the client device 402. For example, the client device 402 receives standard data traffic transmitted by the AP device 412 to the client device 402 via a wireless link that connects the client device 402 and the AP device 412. The client device 402 may obtain channel information based on the data traffic and detect motion of the person 404 based on the channel information.

In some implementations, the wireless signals 410 may include, or may be, broadcast signals transmitted from the AP device 412 and received by the client device 402. As an example, the wireless signals 410 may include, or may be, pings (e.g., Service Set Identifier (SSID) pings) from the AP device 412. In some implementations, the pings are transmitted from the AP device 412 at a rate in a range from about 5 pings per second to about 15 pings per second (e.g., about 10 pings per second). The client device 402 may obtain channel information based on the broadcast signals and detect motion of the person 404 based on the channel information.

In some implementations, the wireless signals 410 may be signals addressed to wireless communication devices, other than the client device 402, that are connected to or associated with the AP device 412. In such implementations, the client device 402 may surreptitiously eavesdrop on transmissions from the AP device 412. The client device 402 may obtains channel information based on the eavesdropped signals and detect motion of the person 404 based on the channel information.

In some implementations, the client device 402 can detect periodic or quasi-periodic changes in the channel information over a series of time points. The series of time points may be included in the time period during which the wireless signals 410 are transmitted. The client device 402 may identify the breathing behavior of the person 404 based on the periodic or quasi-periodic changes. For example, the client device 402 may calculate a breathing rate or another aspect of breathing behavior.

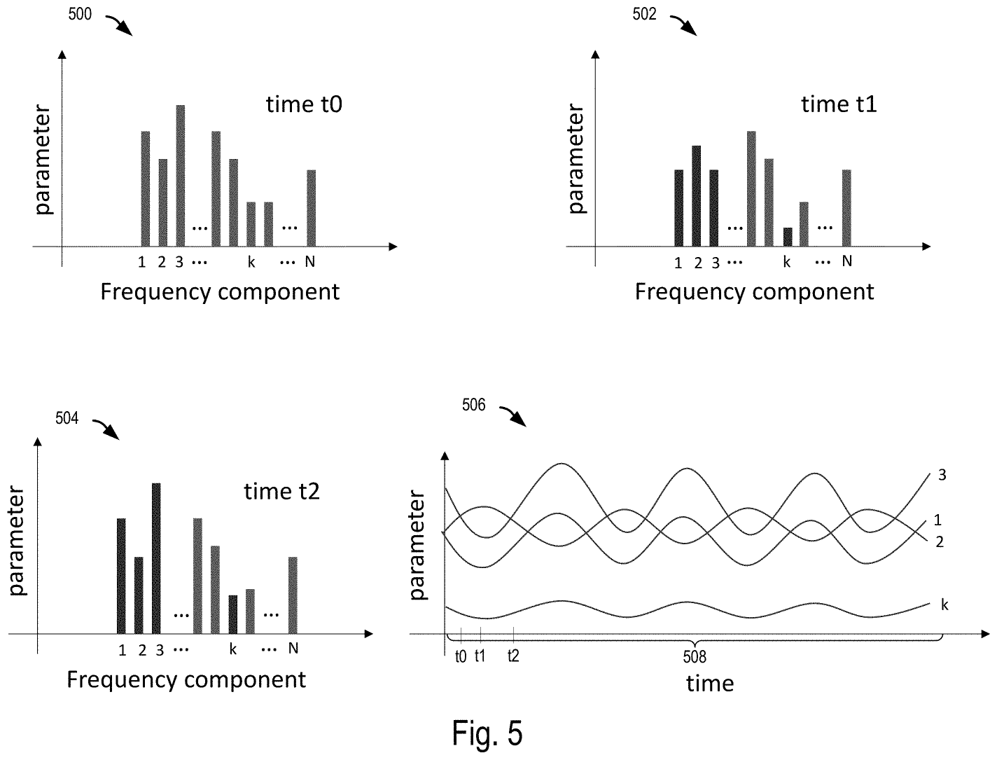

FIG. 5 is a diagram showing example changes in channel information over time. The example changes shown in FIG. 5 can be used by the client device 402 to determine motion data (e.g., the breathing rate of the person 404). In the example of FIG. 5, the channel information includes N frequency components ω₁, ω₂, . . . , ω_{N}, which are indexed on the horizontal axis as frequency components 1 through N.

Plot 500 shows a parameter for each of the frequency components ωₙ of the channel information at a first time point t0 in the series of time points. Plot 502 shows the parameter for each of the frequency components ωₙ of the channel information at a second (later) time point t1 in the series of time points, and plot 504 shows the parameter for each of the frequency components ωₙ of the channel information at a third (later) time point t2 in the series of time points. Plot 506 shows variation in the parameter for some of the frequency components ωₙ of the channel information over the entire series of time points 508.

In some implementations, the channel information for each time point can be, as discussed above in Equation (5), expressed as a complex value Yₙ for a given frequency component ωₙ. The complex value Yₙ can indicate a relative magnitude and phase offset of the received signal at that frequency component ωₙ. In some implementations, the parameter used to determine the breathing rate of the person 404 can be the magnitude of each frequency component (e.g., the magnitude of complex value Yₙ), the power of each frequency component (e.g., the power of complex value Yₙ), the phase of each frequency component (e.g., the phase offset of complex value Yₙ), the magnitude of the real part of each frequency component (e.g., the magnitude of the real part of complex value Yₙ), or the magnitude of the imaginary part of each frequency component (e.g., the magnitude of the imaginary part of complex value Yₙ).

In some instances, the parameter for one or more frequency components ωₙ of the channel information can vary over the series of time points 508 in a periodic or quasi-periodic manner. In some implementations, the variation in the parameter for one frequency component of the channel information over the series of time points 508 may be correlated with the variation in the parameter for another frequency component of the channel information over the series of time points 508. The average rate at which the parameter varies for the correlated frequency components of the channel information can be used to determine the breathing rate of the person 404.

As an illustration, in the example of FIG. 5, the parameter for the frequency components ω₁, ω₂, ω₃, and ωₖ of the channel information varies from time point t0 to time point t1 to time point t2. The parameter for other frequency components ωₙ of the channel information in the example of FIG. 5 is substantially unchanged during time points t0, t1, t2. Viewing the changes in the parameter for the frequency components ω₁, ω₂, ω₃, and ωₖ over the series of time points 508 (e.g., in plot 506) shows that the variations in the parameter for the frequency components ω₁, ω₂, ω₃, and ωₖ are at least quasi-periodic over the series of time points 508. Furthermore, the variations in the parameter for the frequency components ω₁, ω₂, ω₃, and ωₖ are correlated. The average rate at which the parameter varies for the frequency components ω₁, ω₂, ω₃, and ωₖ can be used to determine the breathing rate of the person 404. As an example, an average breathing rate of the person 404 may be in a range from about 7 breaths per minute to about 35 breaths per minute, and the average rate at which the parameter varies for the frequency components ω₁, ω₂, ω₃, and ωₖ may be in a range from about 0.1 Hz to about 0.6 Hz.

As described above, the client device 402 can also determine other types of motion data based on the channel information. For example, the channel information can be used by the client device 402 to determine the sleeping behavior of the person 404 (e.g., the sleep quality or another aspect of sleeping behavior).

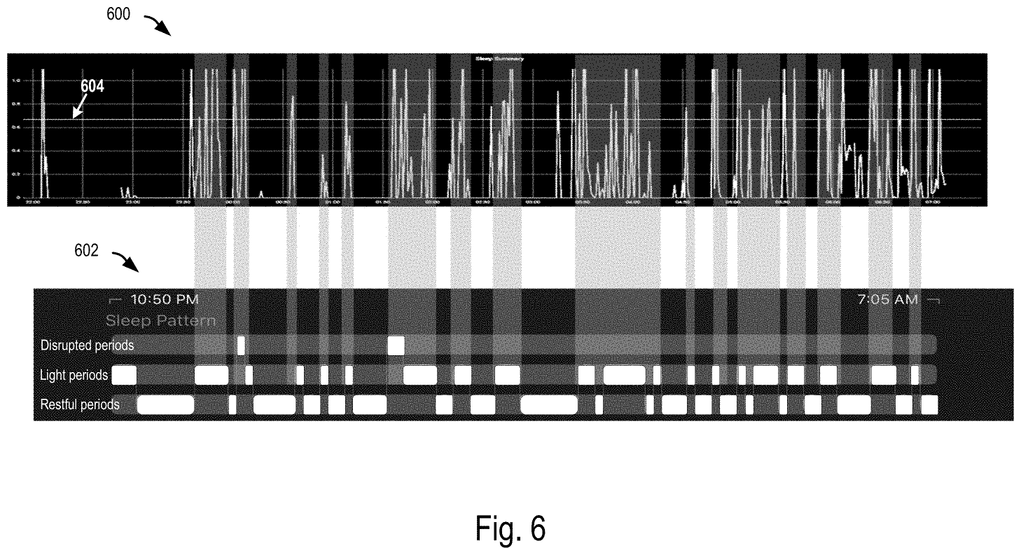

FIG. 6 is a diagram showing a plot 600 of motion data as a function of time and a plot 602 showing corresponding periods of disrupted, light, and restful sleep. The example data shown in FIG. 6 can be provided, for example, by the client device 402 shown in FIG. 4 or by another type of system or device. The horizontal axis in plot 600 represents time (including multiple time points), and the vertical axis represents the degree of motion detected for each time point. The degree of motion for a time point can be represented, for example, as one or more numeric values that indicate the amount of perturbation detected in wireless signals received at the time point; the amount of perturbation can be determined, for example, by analyzing channel information generated from the wireless signals. As shown in FIG. 6, the threshold 604 represents a maximum degree of motion that is indicative of restful sleep. The horizontal axis in plot 602 represents time (including multiple time points) and corresponds to the horizontal axis in the plot 600. In plot 602, three types of sleep patterns are identified: “Disrupted periods”, “Light periods” and “Restful periods”. Other types of sleep patterns may be used. The degree of motion in the plot 600 is used to classify time segments in one of the three sleep patterns. For example, consistent durations with no significant motion above threshold 604 map to “Restful periods,” motion above the threshold 604 for less than a predetermined duration map to “Light periods,” and motion above threshold 604 for greater than a predetermined duration map to “Disrupted periods.”

As an illustration, the person 404 may lie on the bed 408 and place the client device 402 on the nightstand 406. The client device 402 may determine the degree of motion while the person 404 is lying in bed (e.g., based on channel information obtained from wireless signals transmitted from the AP device 412). In some implementations, a low degree of motion may be inferred when the degree of motion is less than a first threshold, and a high degree of motion may be inferred when the degree of motion is greater than a second threshold. As an example, turning or repositioning in the bed 408 can produce a smaller degree of motion over a first duration of time (e.g., between 1 and 5 seconds) compared to instances when the person 404 is walking, which may produce a greater degree of motion over a second (longer) duration of time. In some instances (e.g., the example shown in FIG. 6), the first threshold may be equal to the second threshold, although in other examples the second threshold is greater than the first threshold. In some implementations, the thresholds that are selected can be based on one or more factors, including the degree of the motion that is detected and the duration of the motion that is detected. Furthermore, the thresholds can be selected after user-trials and can also be adjusted automatically by the application that is using the motion detection system on a per-user basis by observing typical over-night behavior of the person 404.

In response to a determination that there is a low degree of motion, the client device 402 may then proceed to determine the average breathing rate of the person 404 to detect whether the person 404 is asleep. In some examples, the average breathing rate of the person 404 may be in a range from about 7 breaths per minute to about 35 breaths per minute while the person 404 is asleep. When the average breathing rate of the person 404 falls within this range and when there is a low degree of motion, the client device 402 may designate a starting time for sleep monitoring (e.g., 10:50 PM in the example of FIG. 6).

The sleeping behavior (e.g., sleep quality) can be determined based on the degree of motion during sleep monitoring. As an example, periods during which the degree of motion is less than the threshold 604 may indicate periods of restful sleep. In some implementations, the client device 402 may continue determining the breathing rate of the person 404 during periods of restful sleep (e.g., during periods of rapid eye movement (REM) sleep). In some examples, the breathing rate of the person 404 may change (e.g., increase) when the person is in restful sleep (e.g., REM sleep).

The person 404 may toss and turn while sleeping. In some examples, when the person 404 starts moving after sleep monitoring has commenced, the client device 402 can stop determining the breathing rate of the person 404 and can instead detect the degree of motion of the person 404. Periods during which the degree of motion is greater than the threshold 604 may indicate either that the person 404 has woken from sleep or that the person 404 is having a period of disrupted or light sleep.

Short bursts of motion occurring after sleep monitoring has commenced may indicate periods of disrupted or light sleep. In some implementations, periods of disrupted or light sleep are detected when the degree of motion is greater than the threshold 604 for a first predetermined duration of time (e.g., less than 5 seconds, or another duration). Conversely, prolonged bursts of motion occurring after sleep monitoring has commenced may indicate that the person 404 has woken from sleep. In some implementations, the client device 402 determines that the person 404 is awake when the degree of motion is greater than the threshold 604 for a second predetermined duration of time (e.g., more than 5 seconds, or another duration). In some implementations, the first and second predetermined durations of time may be functions of the degree of motion detected. For example, a longer duration of time may be associated with a low degree of motion, and a shorter duration of time may be associated with a high degree of motion to distinguish between the light (rapid eye movement) sleep state and the disrupted sleep (awake) state. When the degree of motion indicates that the person 404 has woken from sleep, the client device 402 may designate an ending time for sleep monitoring (e.g., 7:05 AM in the example of FIG. 6). The sleeping behavior (e.g., sleep quality) can be determined based on the level of motion during sleep monitoring. For example, in some implementations, a metric indicative of sleep quality can be determined based on a ratio of a total duration of the periods of restful sleep to the total duration of sleep monitoring (e.g., obtained from the starting and ending times).

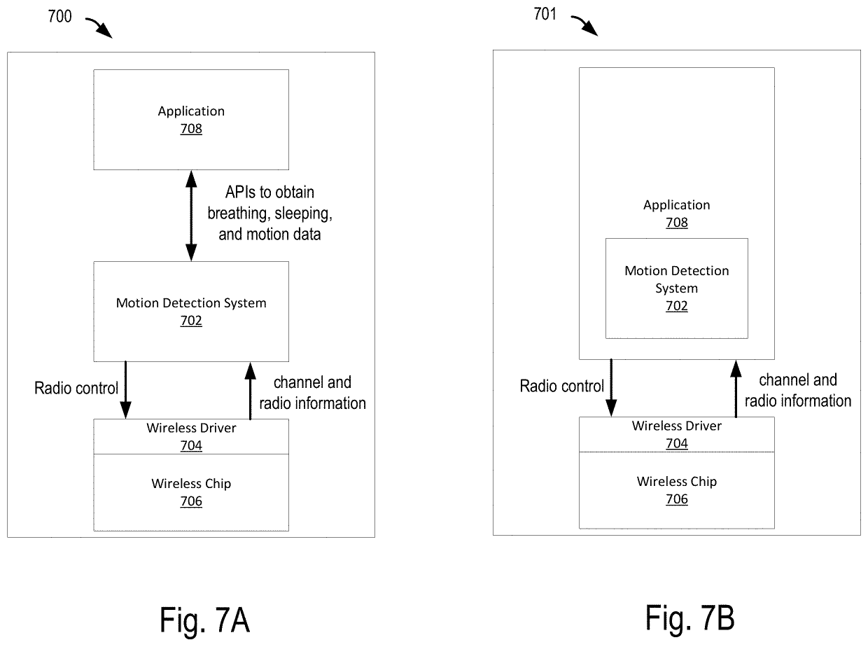

FIGS. 7A and 7B are diagrams showing example implementations of client devices having a motion detection system. FIG. 7A shows a client device 700 having the motion detection system installed as part of the operating system of the client device 700. FIG. 7B shows a client device 701 having the motion detection system installed as part of an application on the client device 701. Each of the client devices 700, 701 may be, for example, identified with the client device 402 shown in FIG. 4 or another type of client device. Furthermore, the motion detection systems shown in FIGS. 7A and 7B may be configured to determine respiratory/breathing activity and monitor sleep quality using the techniques described above.

In the example of FIG. 7A, the client device 700 includes a wireless driver 704. The wireless driver 704 facilitates communication between a wireless chip 706 and the operating system of the client device 700. In the example of FIG. 7A, the motion detection system 702 is installed as part of the operating system core services, and the motion detection systems sends radio control signals to the wireless chip 706 via the wireless driver 704, and receives channel information (e.g., channel state information) and radio information from the wireless chip 706 via the wireless driver 704. The motion detection system 702 determines motion data (e.g., degree of motion, breathing rate, sleeping behavior, or a combination thereof) based on the channel and radio information. An application 708 (e.g., a user application or another type of application) installed on the client device 700 obtains the motion data from the motion detection system 702 via one or more application programming interfaces (APIs). In some implementations, there may be a transfer layer between the one or more APIs and the application 708.

The application 708 can be, for example, a health application, a fitness application, a sleep monitoring application, or another type of application on a smart device. In some cases, the application 708 displays the data, for example, in a graphical user interface or otherwise. In some cases, the application 708 stores the data for long-term data analysis. For instance, the application 708 may store data in the memory of the client device, in the cloud, or elsewhere. In some cases, the application 708 performs further analysis and processing of the data.

In the example of FIG. 7B, the motion detection system 702 is installed as part of the application 708 on the client device 701, and the application 708 communicates with the wireless driver 704 to obtain channel and radio information so that the motion detection system 702 can determine the motion data based on the channel and radio information.

In addition to the examples shown in FIGS. 4 to 6 where the person's breathing rate is determined in the context of sleeping, the client device 700, 701 can also detect the person's breathing rate in instances where the person 404 is not asleep or in bed. As an example, the application 708 may be an application that guides the person 404 to follow a suggested breathing pattern (e.g., breathing depth, rate, and duration). The person 404 can launch the application 708 on the client device 700, 701 to follow the suggested breathing pattern, and the client device 700, 701 can detect the actual breathing pattern of the person 404 as the person 404 attempts to follow the suggested breathing pattern. The motion detection system 702 can detect the actual breathing pattern of the person 404 using the techniques discussed above, compare the suggested breathing pattern to the actual breathing pattern, and provide feedback to the person 404 via a user interface of a user device. In some instances, the feedback can be a confirmation of whether the person 404 correctly followed the suggested breathing pattern. In some instances, the feedback can be an indication of a difference between the suggested breathing pattern and the actual breathing pattern.

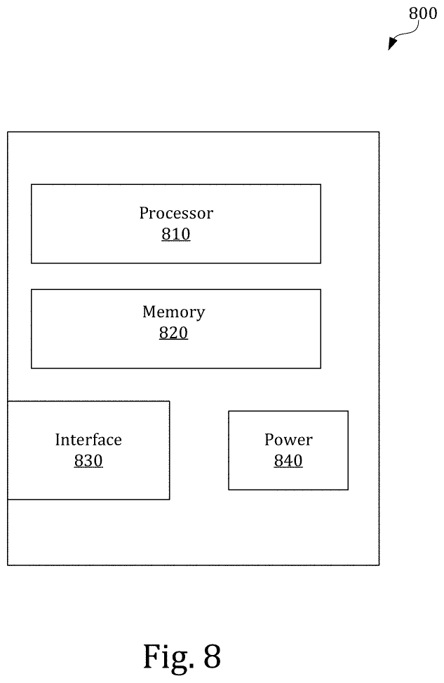

FIG. 8 is a block diagram showing an example wireless communication device 800. As shown in FIG. 8, the example wireless communication device 800 includes an interface 830, a processor 810, a memory 820, and a power unit 840. A wireless communication device (e.g., any of the wireless communication devices 102A, 102B, 102C in FIG. 1) may include additional or different components, and the wireless communication device 1000 may be configured to operate as described with respect to the examples above. In some implementations, the interface 830, processor 810, memory 820, and power unit 840 of a wireless communication device are housed together in a common housing or other assembly. In some implementations, one or more of the components of a wireless communication device can be housed separately, for example, in a separate housing or other assembly.

The example interface 830 can communicate (receive, transmit, or both) wireless signals. For example, the interface 830 may be configured to communicate radio frequency (RF) signals formatted according to a wireless communication standard (e.g., Wi-Fi, 4G, 5G, Bluetooth, etc.). In some implementations, the example interface 830 includes a radio subsystem and a baseband subsystem. The radio subsystem may include, for example, one or more antennas and radio frequency circuitry. The radio subsystem can be configured to communicate radio frequency wireless signals on the wireless communication channels. As an example, the radio subsystem may include a radio chip, an RF front end, and one or more antennas. The baseband subsystem may include, for example, digital electronics configured to process digital baseband data. In some cases, the baseband subsystem may include a digital signal processor (DSP) device or another type of processor device. In some cases, the baseband system includes digital processing logic to operate the radio subsystem, to communicate wireless network traffic through the radio subsystem or to perform other types of processes.

The example processor 810 can execute instructions, for example, to generate output data based on data inputs. The instructions can include programs, codes, scripts, modules, or other types of data stored in memory 820. Additionally or alternatively, the instructions can be encoded as pre-programmed or re-programmable logic circuits, logic gates, or other types of hardware or firmware components or modules. The processor 810 may be or include a general-purpose microprocessor, as a specialized co-processor or another type of data processing apparatus. In some cases, the processor 810 performs high level operation of the wireless communication device 800. For example, the processor 810 may be configured to execute or interpret software, scripts, programs, functions, executables, or other instructions stored in the memory 820. In some implementations, the processor 810 be included in the interface 830 or another component of the wireless communication device 800.

The example memory 820 may include computer-readable storage media, for example, a volatile memory device, a non-volatile memory device, or both. The memory 820 may include one or more read-only memory devices, random-access memory devices, buffer memory devices, or a combination of these and other types of memory devices. In some instances, one or more components of the memory can be integrated or otherwise associated with another component of the wireless communication device 800. The memory 820 may store instructions that are executable by the processor 810. For example, the instructions may include instructions to perform one or more of the operations described above.

The example power unit 840 provides power to the other components of the wireless communication device 800. For example, the other components may operate based on electrical power provided by the power unit 840 through a voltage bus or other connection. In some implementations, the power unit 840 includes a battery or a battery system, for example, a rechargeable battery. In some implementations, the power unit 840 includes an adapter (e.g., an AC adapter) that receives an external power signal (from an external source) and coverts the external power signal to an internal power signal conditioned for a component of the wireless communication device 800. The power unit 820 may include other components or operate in another manner.

Some of the subject matter and operations described in this specification can be implemented in digital electronic circuitry, or in computer software, firmware, or hardware, including the structures disclosed in this specification and their structural equivalents, or in combinations of one or more of them. Some of the subject matter described in this specification can be implemented as one or more computer programs, i.e., one or more modules of computer program instructions, encoded on a computer storage medium for execution by, or to control the operation of, data-processing apparatus. A computer storage medium can be, or can be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially generated propagated signal. The computer storage medium can also be, or be included in, one or more separate physical components or media (e.g., multiple CDs, disks, or other storage devices).

Some of the operations described in this specification can be implemented as operations performed by a data processing apparatus on data stored on one or more computer-readable storage devices or received from other sources.

The term “data-processing apparatus” encompasses all kinds of apparatus, devices, and machines for processing data, including by way of example a programmable processor, a computer, a system on a chip, or multiple ones, or combinations, of the foregoing. The apparatus can include special purpose logic circuitry, e.g., an FPGA (field programmable gate array) or an ASIC (application specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment.. A computer program may, but need not, correspond to a file in a file system. A program can be stored in a portion of a file that holds other programs or data (e.g., one or more scripts stored in a markup language document), in a single file dedicated to the program, or in multiple coordinated files (e.g., files that store one or more modules, sub programs, or portions of code). A computer program can be deployed to be executed on one computer or on multiple computers that are located at one site or distributed across multiple sites and interconnected by a communication network.

Some of the processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application specific integrated circuit).

To provide for interaction with a user, operations can be implemented on a computer having a display device (e.g., a monitor, or another type of display device) for displaying information to the user and a keyboard and a pointing device (e.g., a mouse, a trackball, a tablet, a touch sensitive screen, or another type of pointing device) by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's client device in response to requests received from the web browser.

In a general aspect, a client device is used to monitor sleep based on wireless signals received by the client device.

In some aspects, the client device may be a Wi-Fi client device (e.g., a smartphone or wearable device like a smartwatch). In some aspects, an access point device in the space does not have a motion detection system installed thereon, therefore allowing the access point device to be dedicated for providing other functions (e.g., wireless access to the client device and to other wireless-enabled devices in the space). In some aspects, the access point device provides Wi-Fi access point capabilities through SSID broadcasts that can be used by the client device as a source for channel information. In some instances, the motion detection system can be part of an application on the client device or the client device's operating system. The motion detection system may access channel information (e.g., Wi-Fi channel state information data) provided by the radio firmware of the client device to sense motion in the space.

In a first example, a method includes receiving, at a wireless communication device (e.g., a smartphone or a smart watch, or another type of device) operating as a client in a wireless communication network (e.g., a wireless mesh network or another type of wireless local area network), wireless signals transmitted through a space from an access point of the wireless communication network. The first wireless signals are received over a first time period. The method further includes, by operation of the client device, generating channel information (e.g., channel responses) from the wireless signals, processing the first channel information to identify a degree of motion in the space during the first time period, and processing the channel information to identify an average breathing rate of a person in the space during the first time period (e.g., as shown and described with respect to FIGS. 4, 5, 6). After determining that the degree of motion is below a first threshold and that the average breathing rate is below a second threshold, a sleep monitoring process is initiated (e.g., as shown and described with respect to FIGS. 4, 5, 6). The sleep monitoring process includes receiving, at the wireless communication device, additional wireless signals transmitted through the space, wherein the additional wireless signals are received over a second time period (e.g., a later time period); generating second channel information from the additional wireless signals; and processing the second channel information to identify a category of sleep experienced by the person during the second time period (e.g., as shown and described with respect to FIGS. 4, 5, 6). For instance, “Disrupted periods”, “Light periods,” and “Restful periods” of sleep may be identified as discussed above with respect to FIG. 6, or other types of sleep categories may be identified.

In a second example, a wireless communication device operating as a client in a wireless communication network includes a wireless communication interface, one or more processors, and memory storing instructions that are operable to perform one or more operations of the first example. In a third example, a computer-readable medium stores instructions that are operable, when executed by data processing apparatus, to perform one or more operations of the first example.

Implementations of the first, second, or third example may include one or more of the following features. Processing the second channel information to identify a category of sleep can include processing the second channel information to identify a second degree of motion in the space during the second time period; comparing the second degree of motion with threshold values associated with a respective plurality of sleep categories; and identifying the category of sleep based on the comparison. The plurality of sleep categories can include a first category of sleep that is identified if the second degree of motion is below a third threshold, a second category of sleep that is identified if the second degree of motion is above the third threshold and below a fourth threshold, and a third category of sleep that is identified if the second degree of motion is above the fourth threshold (e.g., as shown and described with respect to FIG. 6). The sleep monitoring process can include receiving, at the wireless communication device, third wireless signals transmitted through the space, in which the third wireless signals are received over a third time period; generating third channel information from the third wireless signals; processing the third channel information to identify a degree of motion in the space during the third time period; and terminating the sleep monitoring process in response to a determination that the degree of motion is above a third threshold (e.g., designating an ending time for sleep monitoring, as discussed above). The second channel information can be processed to identify a category of sleep that includes identifying multiple categories of sleep during the second time period, in which the multiple categories of sleep are associated with respective time segments within the second time period (e.g., as shown and described with respect to FIG. 6 or otherwise). A graphical representation can be generated to represent the multiple categories of sleep associated with the respective time segments (e.g., as shown in FIG. 6 or otherwise), and the graphical representation can be displayed on a display component of the wireless communication device. The first and second threshold can be determined by the mobile communication device. The sleep monitoring process can be performed by a motion detection system (e.g., a motion detection software module) in an operating system installed on the wireless communication device (e.g., as shown in FIG. 7A). The sleep monitoring process can be performed by a motion detection system (e.g., a motion detection software module) in an application installed on the wireless communication device (e.g., as shown in FIG. 7B).

While this specification contains many details, these should not be understood as limitations on the scope of what may be claimed, but rather as descriptions of features specific to particular examples. Certain features that are described in this specification or shown in the drawings in the context of separate implementations can also be combined. Conversely, various features that are described or shown in the context of a single implementation can also be implemented in multiple embodiments separately or in any suitable subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single product or packaged into multiple products.

A number of embodiments have been described. Nevertheless, it will be understood that various modifications can be made. Accordingly, other embodiments are within the scope of the description above.

What is claimed is:

1. A method comprising:
receiving, at a wireless communication device operating as a client in a wireless communication network, first wireless signals transmitted through a space from an access point of the wireless communication network, wherein the first wireless signals are received over a first time period;
by operation of one or more processors of the wireless communication device:
generating first channel information from the first wireless signals;
processing the first channel information to identify a degree of motion in the space during the first time period;
processing the first channel information to identify an average breathing rate of a person in the space during the first time period; and
initiating a sleep monitoring process in response to a determination that:
the degree of motion is below a first threshold; and
the average breathing rate is below a second threshold;
performing the sleep monitoring process on the wireless communication device, wherein the sleep monitoring process comprises:
receiving, at the wireless communication device, second wireless signals transmitted through the space, wherein the second wireless signals are received over a second time period; and
by operation of the one or more processors of the wireless communication device:
generating second channel information from the second wireless signals; and
processing the second channel information to identify a category of sleep during the second time period.

2. The method of claim 1, wherein the degree of motion is a first degree of motion and processing the second channel information to identify a category of sleep comprises:
processing the second channel information to identify a second degree of motion in the space during the second time period;
comparing the second degree of motion with threshold values associated with a plurality of sleep categories; and
identifying the category of sleep based on the comparison.

3. The method of claim 2, wherein the plurality of sleep categories includes:
a first category of sleep that is identified if the second degree of motion is below a third threshold;
a second category of sleep that is identified if the second degree of motion is above the third threshold and below a fourth threshold; and
a third category of sleep that is identified if the second degree of motion is above the fourth threshold.

4. The method of claim 2, wherein the sleep monitoring process comprises:
receiving, at the wireless communication device, third wireless signals transmitted through the space, wherein the third wireless signals are received over a third time period; and
by operation of the one or more processors of the wireless communication device:
generating third channel information from the third wireless signals; and
processing the third channel information to identify a third degree of motion in the space during the third time period; and
terminating the sleep monitoring process in response to a determination that the third degree of motion is above a third threshold.

5. The method of claim 1, wherein processing the second channel information to identify a category of sleep comprises identifying multiple categories of sleep during the second time period, wherein the multiple categories of sleep are associated with respective time segments within the second time period.

6. The method of claim 5, comprising:
generating a graphical representation of the multiple categories of sleep associated with the respective time segments; and
displaying the graphical representation on a display component of the wireless communication device.

7. The method of claim 1, wherein the first threshold and the second threshold are determined by the wireless communication device.

8. The method of claim 1, wherein the first channel information is processed to identify an average breathing rate of a person in the space during the first time period in response to a determination that the degree of motion is below the first threshold.

9. The method of claim 1, wherein at least a portion of the sleep monitoring process is performed by a motion detection system that is included in an operating system installed on the wireless communication device.

10. The method of claim 1, wherein at least a portion of the sleep monitoring process is performed by a motion detection system that is included in an application installed on the wireless communication device.

11. A wireless communication device comprising:
a wireless communication interface;
one or more processors; and
memory storing instructions that are operable when executed by the one or more processors to perform operations comprising:
generating first channel information from first wireless signals, wherein the first wireless signals are:
transmitted through a space from an access point of a wireless communication network, and
received, over a first time period, by the wireless communication interface of the wireless communication device operating as a client in the wireless communication network;
processing the first channel information to identify a degree of motion in the space during the first time period;
processing the first channel information to identify an average breathing rate of a person in the space during the first time period; and
initiating a sleep monitoring process in response to a determination that:
the degree of motion is below a first threshold; and
the average breathing rate is below a second threshold;
performing the sleep monitoring process on the wireless communication device, wherein the sleep monitoring process comprises:
generating second channel information from second wireless signals, wherein the second wireless signals are:
transmitted through the space, and
received, over a second time period, by the wireless communication interface of the wireless communication device; and
processing the second channel information to identify a category of sleep during the second time period.

12. The wireless communication device of claim 11, wherein the degree of motion is a first degree of motion and processing the second channel information to identify a category of sleep comprises:
processing the second channel information to identify a second degree of motion in the space during the second time period;
comparing the second degree of motion with threshold values associated with respective a plurality of sleep categories; and
identifying the category of sleep based on the comparison.

13. The wireless communication device of claim 12, wherein the plurality of sleep categories includes:
a first category of sleep that is identified if the second degree of motion is below a third threshold;
a second category of sleep that is identified if the second degree of motion is above the third threshold and below a fourth threshold; and
a third category of sleep that is identified if the second degree of motion is above the fourth threshold.

14. The wireless communication device of claim 12, wherein the sleep monitoring process comprises:
generating third channel information from the third wireless signals, wherein the third wireless signals are:
transmitted through the space, and
received, over a third time period, by the wireless communication interface of the wireless communication device;
processing the third channel information to identify a third degree of motion in the space during the third time period; and
terminating the sleep monitoring process in response to a determination that the third degree of motion is above a third threshold.

15. The wireless communication device of claim 11, wherein processing the second channel information to identify a category of sleep comprises identifying multiple categories of sleep during the second time period, wherein the multiple categories of sleep are associated with respective time segments within the second time period.

16. The wireless communication device of claim 15, comprising a display component operable to display a graphical representation of the multiple categories of sleep associated with the respective time segments.

17. The wireless communication device of claim 11, wherein the wireless communication device comprises an installed operating system that includes a motion detection system, and at least a portion of the sleep monitoring process is performed by the motion detection system.

18. The wireless communication device of claim 11, wherein the wireless communication device comprises an installed application that includes a motion detection system, and at least a portion of the sleep monitoring process is performed by the motion detection system.
